(19) United States
(12) Patent Application Publication
(10) Pub. No.: US 2025/0099932 A1
(43) Pub. Date: Mar. 27, 2025

(54) Material Mixing Cartridge for Dispensing Systems

(71) Applicant: Cellink Bioprinting AB, Göteborg (SE)

(72) Inventors: Mateusz Piotrzkowski, Göteborg (SE); Anders Dahl, Göteborg (SE); Ginger Lohman, Göteborg (SE); Erik Gatenholm, Göteborg (SE); Hector Martinez, Göteborg (SE)

(21) Appl. No.: 18/730,968

(22) PCT Filed: Jan. 24, 2023
(86) PCT No.: PCT/SE2023/050061

§ 371 Date: Jul. 22, 2024

(30) Foreign Application Priority Data
Jan. 28, 2022 (SE) 2250084-7

(51) Int. Cl. B01F 35/88; B01F 35/221; B01F 35/75; B01L 3/02; B33Y 30/00

(52) U.S. Cl. CPC B01F 35/883; B01F 35/2215; B01F 35/75425; B01L 3/0241; B01L 3/0293; B33Y 30/00

(57) ABSTRACT

A material mixing cartridge for a dispensing system comprises a dispensing chamber, with a material dispensing outlet and a dispensing actuator for dispensing material through the material dispensing outlet. The mixing cartridge further comprises at least two material compartments for receiving material to be mixed, and at least one material transfer channel which is adapted for material transfer between the at least two material compartments and the dispensing chamber The mixing cartridge further comprises at least one valve arranged to regulate material flow between a material compartment and the dispensing chamber via the at least one material transfer channel.

### FIELD OF THE INVENTION

The present invention relates to a material mixing cartridge, and in particular to a material mixing cartridge for dispensing systems, and a method of use of such a material mixing cartridge, according to the preambles of the independent claims.

### BACKGROUND

3D bioprinting, i.e. 3D printing using biomaterials, is an increasingly expanding field. Similarly, other related dispensing processes of biomaterials, such as biomaterial extrusion systems or droplet dispensing systems capable of reproducible dispensing of controlled volumes of fluid material, are becoming more and more common.

However, many challenges exist, especially in regard to printing or dispensing material that requires specific environmental and handling conditions. For instance, biomaterials are normally maintained and printed at temperatures much lower than traditional 3D printing. Biomaterial, including material containing biological material, such as living cells, must be used and handled such that viability and sterility is maintained. In addition, materials normally need to be mixed prior to and/or during the printing or dispensing process. Present workflows require users to perform all material preparation steps before conducting a 3D bioprinting or droplet dispensing process. For example, users need to mix the bioink with cells in medium. For some hydrogels used as bioinks in bioprinting it is also necessary to perform additional preparation steps such as neutralizing collagen.

Performing material preparation steps is inconvenient and time-consuming for the users. Furthermore, it can be a source of errors. If cells in medium aren't mixed thoroughly enough with the ink, the homogeneity of the solution will lead to different number of cells throughout the printed model. If the user mixes the fluids too thoroughly, a lot of stress will be put on cells and their viability will suffer.

Neutralizing collagen is problematic because the initial pH of the material varies from batch to batch. Therefore, the amount of NaOH needed to adjust the pH will vary, and the user needs to check the pH before adding cells.

Even though mixing has been performed prior to printing, when the user places a cartridge filled with bioink with cells into a printhead of a bioprinter or a dispensing unit of a dispensing system, cell sedimentation starts to occur. As a result, samples that are dispensed first contain more cells than samples extruded at the end of the process.

Furthermore, some bioinks require to be mixed with cells in medium at a certain temperature. E.g., collagen should be kept around 4 degrees C., otherwise it will self-assemble. With current methods it's difficult to maintain the temperature of consumables and ink. Existing solutions of mixing biomaterials before 3D printing or other dispensing include double-barreled syringe solutions such as that shown in e.g. CN209940978U or

CN106891527A, wherein the content of two different syringe barrels are pushed into a common static mixer. Another example of a dispensing nozzle is shown in W003017745A2, where various types of mixers are employed in a tip chamber. Limitations of such solutions include large dead volume in the various channels and mixer parts, fixed ratios of material and no possibility of resuspending the mixed material.

The inventors of the present invention has thus identified a need for an improved material mixing cartridge, specifically suitable for biomaterials, including cellular material and living cells.

### SUMMARY

An object of the present disclosure is to provide a material mixing cartridge which enables quick and precise preparation of the material needed for a bioprinting or dispensing process. A further object is to provide a material mixing cartridge which allows flexibility in ratios, volumes and timing of different materials to be mixed.

Yet another object is to provide a material mixing cartridge which provides periodical mixing of the material during a dispensing process to ensure a homogenous dispersion of cells in a printed construct.

The above-mentioned objects and other advantages are achieved by the present disclosure according to the independent claims. Preferred embodiments are set forth in the dependent claims.

A material mixing cartridge for a dispensing system comprises a dispensing chamber, with a material dispensing outlet and a dispensing actuator for dispensing material through the material dispensing outlet. The mixing cartridge further comprises at least two material compartments for receiving material to be mixed, and at least one material transfer channel which is adapted for material transfer between the at least two material compartments and the dispensing chamber The mixing cartridge further comprises at least one valve arranged to regulate material flow between a material compartment and the dispensing chamber via the at least one material transfer channel. The dispensing chamber, material compartments and at least one valve are configured such that material transfer between a material compartment and the dispensing chamber may be performed independently of dispensing material through the material dispensing outlet. By independently is meant that the material transfer may be performed independently in time of any dispensing action, for example before during or after the dispensing of material from the material dispensing outlet.

Further, the dispensing chamber, material compartments and at least one valve are configured such that any material in the dispensing chamber may be transferred to any material compartment. In other words, material may be transferred from the material compartments to the dispensing chamber, and thereafter back to any material compartment, and once again to the dispensing chamber before being dispensed. This provides a gentle but thorough mixing of the different materials before dispensing the desired mixture. In addition, the mixing by transfer back and forth between material compartments and the dispensing chamber may be performed once or several times. In addition, the mixing by transfer back and forth between material compartments and the dispensing chamber may be performed before and/or during the dispensing through the material dispensing outlet, i.e. independently in time.

The disclosed material mixing cartridge makes it possible to automatically prepare the bioink and then to mix the ink with cells in medium, all within the material mixing cartridge itself.

Further a method for mixing material to be dispensed in a dispensing system comprises the steps of
a) adding material to a first material compartment in a material mixing cartridge for a dispensing system, said material compartment being connected to a dispensing chamber in said material mixing cartridge via a material transfer channel,
b) adding material to a second material compartment in said material mixing cartridge, said second material compartment also being connected to said dispensing chamber in said material mixing cartridge via a material transfer channel, said material mixing cartridge further comprising at least one valve arranged to regulate material flow between at least one of the material compartments and the dispensing chamber,
c) transferring material from the first material compartment to the dispensing chamber via the connected transfer channel,
d) subsequently or simultaneously transferring material from the second material compartment to the dispensing chamber via the connected transfer channel,
e) transferring material from the dispensing chamber back to one of the material compartments and thereafter from said material compartment to the dispensing chamber via the transfer channel,
f) dispensing the mixed material from the dispensing chamber through a material dispensing outlet.

Further aspects and advantages of material mixing cartridges are described below.

### BRIEF DESCRIPTION OF THE DRAWINGS

FIG. 1 is a schematic illustration of a first aspect of a material mixing cartridge.

FIG. 2 is a schematic illustration of another aspect of a material mixing cartridge.

FIG. 3 is a schematic illustration of yet another aspect of a material mixing cartridge.

FIG. 4 is a schematic illustration of a dispensing system wherein a material mixing cartridge is mounted.

FIG. 5 is a schematic illustration of a valving system in a material mixing cartridge.

### DETAILED DESCRIPTION

The term “materials” described herein for use in a material mixing cartridge may include biomaterials and related material, for example, materials used to supplement the bioprinting process. Herein, the term “biomaterial” encompasses all material, compositions and agents suitable for and used in bioprinting and dispensing systems in a laboratory or healthcare setting. Similarly, the material mixing cartridge disclosed is intended to be used to mix such materials with each other for use in a dispensing system. Non-limiting examples of biomaterials include. bioinks, hydrogels, growth medium, material containing biological material, such as living cells, various reagents etc. Non-limiting examples of non-biomaterials are support material and sacrificial inks, gels and other materials that are used to supplement the bioprinting process.

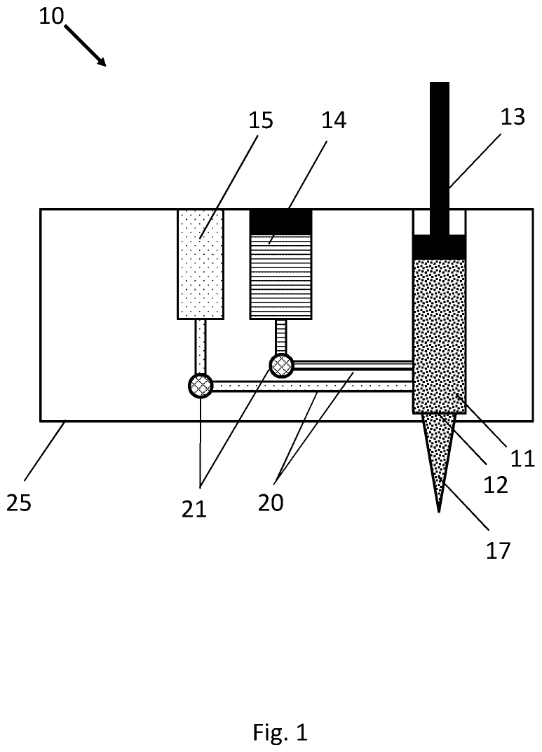

FIG. 1 shows a schematic view of a first aspect of a material mixing cartridge for dispensing systems. A material mixing cartridge 10 comprises a dispensing chamber 11. The dispensing chamber is adapted for dispensing mixed material out of the cartridge and thus has a material dispensing outlet 12 and a dispensing actuator 13 for dispensing material through the material dispensing outlet 12. The dispensing actuator 13 may be a piston arranged in the dispensing chamber, as illustrated in the figures, or any other suitable pressure applicator. Thus, various alternatives to actuate dispensing are conceivable, such as piston extrusion, syringe extrusion, pressure based extrusion, electro-magnetic extrusion or droplet jetting.

The material mixing cartridge 10 is preferably provided with a fixed or attachable dispensing nozzle 17. Such a nozzle may be attached via a Luer Lock or a different type of connector. Further, the nozzle 17 may be provided with a nozzle cap that will help preserve the sterility of the nozzle when transferring the cartridge 10 from a sterile environment to a dispensing instrument. Such a nozzle cap is intended to be removed before dispensing.

The material dispensing outlet 12 is preferably provided with a suitable valve (not shown in the Figures), configured such that the outlet 12 may be closed during a mixing procedure.

The dispensing system is preferably a 3D bioprinter or a droplet dispensing system, and the material mixing cartridge is preferably adapted for direct mounting in the dispensing system. In other aspects, the dispensing system may be a manual dispensing device or system.

The material mixing cartridge 10 is further provided with at least two material compartments for receiving material to be mixed. In FIG. 1 there are two material compartments 14, 15. For example, the first material compartment 14 may be intended for receiving a bioink or hydrogel for 3D bioprinting. The second material compartment 15 may be intended for receiving a medium containing cells. In bioprinting, it is common to mix cells in medium with a bioink, and use the mixed material for bioprinting a 3D construct. The cartridge thus comprises at least two material compartments for substrates, i.e. initial materials to be used in a bioprinting or dispensing process. In one example, one of those compartments may be intended for a bioink and another may be intended for cells in medium. In an alternative implementation one of those compartments may be intended for a bioink and another may be intended for a reagent.

Each material compartment is provided with a material transfer channel 20 arranged to fluidly connect with the dispensing chamber 11. The transfer channels 20 are thus adapted for material transfer between each of the material compartments 14, 15 and the dispensing chamber 11. Having separate material transfer channels for each material compartment makes it easy to control the amount of each material individually. However, in other aspects, as detailed below, at least part of a material transfer channel 20 may be common to two or several material compartments.

Each material compartment is further preferably provided with a valve 21 arranged to regulate material flow between the respective material compartment and the dispensing chamber via the material transfer channel. In the present aspect, each material compartment is provided with a valve 21 arranged along or in connection to the material transfer channel 20. However, a valve 21 may also be arranged at other positions along the path from the material compartment to the dispensing chamber. It is also conceivable that the material compartments are provided with common valves in a suitable arrangement, wherein the transfer of material from two or more compartments may be controlled by actuating a common valve.

The valves 21 may be of any suitable type, and may be individually or commonly regulated. Valves may be actuated in sequence to supply different compositions and material distribution before or after mixing. Preferably, each of the valves 21 are arranged to be movable between at least i) a closed configuration, ii) a configuration allowing material flow from the connected material compartment to the dispensing chamber, and, optionally, iii) a configuration allowing material flow from the dispensing chamber to the connected material compartment. In some cases, depending on the type of valve, configuration ii) and iii) are the same, i.e. a configuration open in both directions.

The dispensing chamber, material compartments and at least one valve are configured such that material in the dispensing chamber may be transferred back and forth to at least one of the material compartments for mixing of the material. Moreover, the arrangement allows material transfer between a material compartment and the dispensing chamber to be performed independently of dispensing material through the material dispensing outlet, as will be described in detail below. Thus mixing of the material is separate from the dispensing procedure.

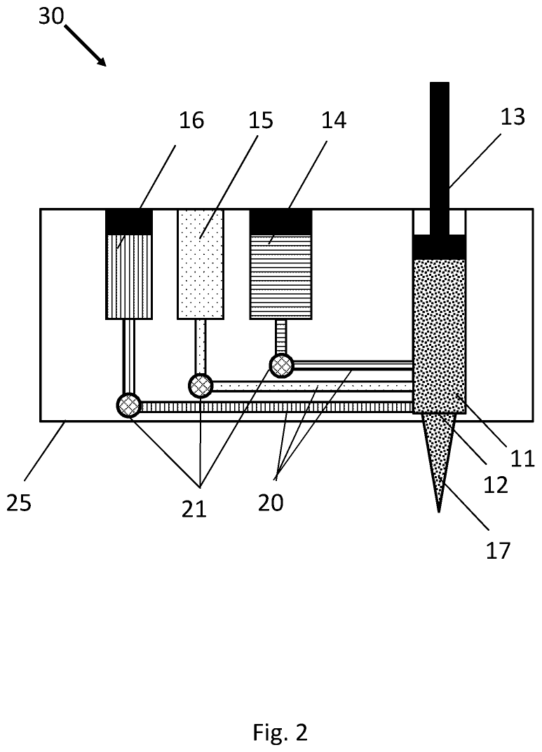

FIG. 2 illustrates another aspect of a material mixing cartridge. The material mixing cartridge 30 comprises all the features of the material mixing cartridge 10 described above in connection to FIG. 1. In addition, the material mixing cartridge 30 has a third material compartment 16, and a dedicated material transfer channel 20 adapted for material transfer between the material compartment 16 and the dispensing chamber 11. A material mixing cartridge may comprise a fourth, fifth or further material compartments (not illustrated), similarly connected to the dispensing chamber, allowing mixture of any specified number or combination of materials.

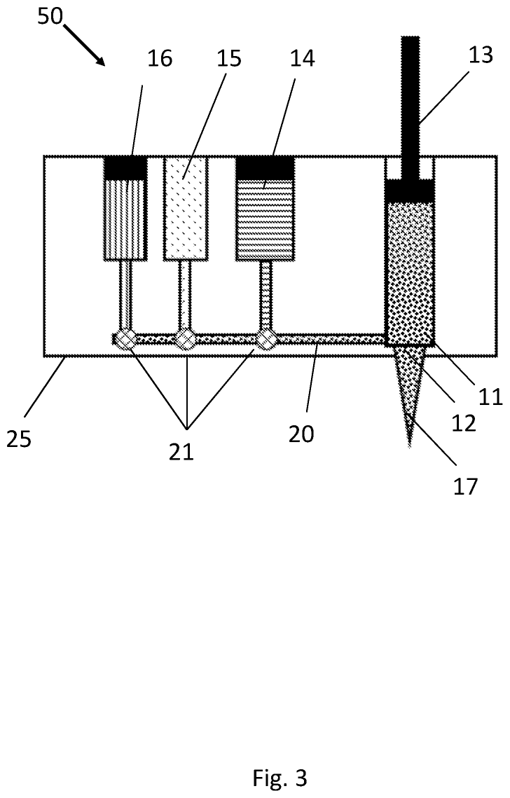

FIG. 3 illustrates yet another aspect of a material mixing cartridge. The material mixing cartridge 50 comprises essentially all the features of the material mixing cartridge 10 or 30 described above in connection to FIGS. 1 and 2. However, in this aspect the different material compartments connect to a common material transfer channel 20 connected to the dispensing channel. Using an at least partly common material transfer channel may reduce overall dead volume in the cartridge, resulting in less loss of material during a mixing and dispensing procedure. Furthermore, a common transfer channel may allow flushing out of any remaining material in the channel, using the most cost efficient of the materials, thus not using more of a high cost material than necessary.

As previously indicated, the dispensing chamber, material compartments and at least one valve are configured such that material may be transferred in either direction between the dispensing chamber and any material compartment. In any of the material mixing cartridges disclosed herein, each material compartment 14, 15, 16 may be connected to a material transfer channel 20 via an individual valve 21 for regulating flow between each material compartment 14, 15, 16 and the dispensing chamber 11. This allows for precise regulation of when and how much material is transferred to or from the relevant material compartment. Thus, each material transfer may be controlled independently, and may be performed, for example, before and/or during dispensing through the material dispensing outlet 12. Further, material from different material compartments 14, 15, 16 may be transferred at the same time or separately to the dispensing chamber 11.

The separation of the mixing step from the dispensing of a mixture also allows mixing of materials in any desired sequence and/amount, and can vary over time during a printing procedure. Thus, it is conceivable to use a series of different mixing steps, e.g. with different materials and/or different ratios of materials from the material compartments 14, 15, 16 interspersed with dispensing steps. This allows dispensing of varying compositions from the material mixing cartridge during the timeline of a dispensing procedure.

In use, two or more different materials to be mixed are added to the material compartments 14, 15, 16. As an example, a bioink is added to the first material compartment 14. Such bioink or other material may be supplied in a sealed capsule, and when the capsule is arranged in the material compartment the received capsule is opened to the material transfer channel, e.g. by breaking of a seal. For example, a capsule connects to the cartridge via a leak proof connection and the action of connecting the capsule breaks a seal/foil in the capsule causing it to open. As an alternative, the first material may be added to the first material compartment 14 by pipetting, syringe transfer or other suitable means.

Thereafter, a second material is added to the second material compartment 15. As an example, cells in medium are added. These may be added e.g. by pipetting, syringe transfer or other suitable means. In some aspects, a third and optionally any subsequent material is added to a respective third, fourth etc. material compartment.

Preferably, each material compartment may be closed and/or sealed after material is added, preferably using a suitable cap or cover.

After relevant materials have been added to the material compartments, material is transferred from each material compartment to the dispensing chamber 11. This may be achieved either in sequence, stepwise or simultaneously. During transfer, the relevant valves 21 are actuated to the suitable configuration. Furthermore, during transfer of material from a material compartment to the dispensing chamber, the material dispensing outlet 12 is preferably manually or automatically closed, e.g. by use of a suitable valve, to avoid dispensing material through the dispensing outlet 12 during mixing of materials, i.e. before proper mixing of material has been performed.

In some aspects, each material compartment 14, 15, 16 may be provided with a transfer actuator, such as a piston or other pressure source, adapted to provide pressure within the respective material compartment such that material may be transferred from the material compartment to the material transfer channel 20, and further to the dispensing chamber 11. In other aspects, transfer of material, when the appropriate valve 21 is open, may be achieved by aspiration through the channels, e.g. by capillary action, or by a negative pressure being applied in the dispensing chamber, for example by moving the dispensing piston 13 upwards, i.e. expanding the volume in the dispensing chamber.

Thus, fluid materials, such as cells in cell medium, can be pushed to different compartments of the cartridge by means of a positive or a negative air pressure or by a mechanical movement generated by the instrument. Fluid materials can be pushed from a capsule by the means of air pressure or a mechanical movement generated by the instrument. Materials from different compartments are transferred to the dispensing chamber by either external pressure or aspiration. Depending on the viscosity of certain bio-inks, only aspirating may not create a sufficient pressure differential to move the fluid so an external pressure can be applied to the Ink capsule compartment. Especially for viscous fluids, a transfer actuator is needed in order to transfer material through the material transfer channel 20.

Some mixing of the materials will occur by simply combining the different materials in the dispensing chamber. However, in many cases, this mixing is not enough, as different materials may have different tendencies to mix well, and sedimentation and/or layering of material may occur. Therefore a thorough further mixing is oftentimes needed, to ensure good mixing before dispensing material. By transferring the material from one chamber, through an elongated channel and into another chamber, turbulence is created, promoting thorough mixing of the materials.

When using the disclosed material mixing cartridge, mixing is performed, after all required materials are transferred into the dispensing chamber, by transferring material from the dispensing chamber back to one of the material compartments and thereafter from the material compartment to the dispensing chamber via the transfer channel. This process may be repeated a desired number of times, to ensure thorough mixing before dispensing the material. Transfer from the dispensing chamber may be performed by applying pressure, e.g. by piston 13, in the dispensing chamber, while maintaining a closed dispensing outlet 12, thus pushing the mixture back into channel 20. As an example, the mixture may be directed into the material compartment 14 by actuating the relevant valves 21, and thereafter transferred back to the dispensing chamber via the transfer channel 20. This may be repeated until a satisfactory homogenous mixture is achieved.

Mixing material, especially biomaterial containing living cells, in the described manner has shown to be effective and still gentle enough so as to not damage the cells unnecessarily. By providing essentially smooth channels and compartments, less shear forces will be applied to the mixture, causing less cell damage compared to other types of mixers. Furthermore, mixing the entire volume of material contained in at least one of the material compartments, such as a bioink in material compartment 14, and thereafter being able to dispense the entire mixed volume of material, will ensure minimal loss of material overall.

When the material is suitably mixed, the mixture is dispensed from the dispensing chamber through the material dispensing outlet 12, by e.g. opening the material dispensing outlet 12, closing the valves 21 and actuating the piston 13.

Notably, in the mixing cartridges disclosed herein, mixing and dispensing are not optimally performed simultaneously, and in fact, should preferably be done separately. Hence, the mixing cartridges disclosed herein are adapted such that mixing of material is performed separately from the dispensing step. Materials may be transferred from the material compartments simultaneously or in any desired sequence, for example one at a time, or first two materials, thereafter a third etc. Mixing may be performed when any combination of materials are present in the dispensing chamber. In some aspects, a first combination of materials is mixed as described above, and thereafter another or further materials transferred to the mixture in the dispensing chamber, followed by another mixing procedure. Only when the final desired material mixture for dispensing is present in the dispensing chamber, the dispensing procedure is executed, e.g. by activating a dispensing piston in the dispensing chamber.

As an optional step, the mixture may be remixed anytime during a dispensing process, either at set intervals or manually when needed. This will help keep e.g. cells suspended, and the dispensed mixture homogenous throughout a dispensing process. Thus, it is conceivable that after a mixed material is partly dispensed, dispensing may be paused, a mixing procedure performed by transferring mixture back and forth to a material compartment a desired number of times, and thereafter the dispensing is resumed. This ensures that a dispensed mixture is well mixed throughout the time it takes to complete a dispensing procedure. This is especially important when using cells suspended in medium, to counteract cell sedimentation in the dispensing chamber.

Another example is that after a mixed material is partly dispensed, dispensing may be paused, and either a further material or more of one material is added to the mixture by transferring from a material compartment to the dispensing chamber. Thereafter a mixing procedure is performed by transferring mixture back and forth to a material compartment a desired number of times, and the dispensing is resumed. Using such a procedure allows dispensing to produce a gradient regarding concentration and/or composition of a mixed material in the printed construct or dispensed product.

The disclosed material mixing cartridge and disclosed method of use provides flexibility in both proportions and timing of mixing of materials, as the mixing procedure is separated from the dispensing procedure both in space and time. Thus, material can be applied both independently of each other and/or in different ratios to the dispensing chamber, and thereafter mixed. Once initial mixing is performed, dispensing may be executed. Furthermore, mixing may be performed during a dispensing procedure, or at specific time intervals, to e.g. resuspend cells in the solution, thereby ensure a homogenous dispensed material over time. In addition, very precise amounts of material may be transferred from each material compartment, resulting in less loss of material overall. The cartridge also has very little dead volume.

The material mixing cartridge disclosed herein is preferably provided with an outer housing 25 enclosing the cartridge, such that the dispensing chamber 11, material compartments 14, 15, 16, material transfer channel 20 and at least one valve 21 are contained within the outer housing. Such housing provides a practical enclosure for handling of the cartridge, as well as for mounting it in a dispensing system. Further the arrangement helps keep the materials contained and preferably sterile throughout the procedure.

The cartridge may be made of a polymer, metal, glass, elastomer or a combination of those materials.

A material mixing cartridge may further comprise a temperature regulation means for controlling temperature of the material in the cartridge, either for controlling the temperature of the entire cartridge, or at least the dispensing chamber. The cartridge may alternatively be adapted such that either the entire cartridge, or at least the dispensing chamber is temperature controlled by a temperature-regulated block at a dedicated cartridge station, which will be described further below.

The material compartments of the material mixing cartridges disclosed herein may have a volume in the range of approximately 500 μl to 10,000 μl, more preferably in the range of 1500 μl to 5000 μl. The total mixed volume capacity of the cartridge may be in the range of 2000 μl to 10,000 μl, more preferably in the range of 3000 μl to 7000 μl.

A material mixing cartridge 10, 30, 50 is preferably provided as a single-use and sterilized device. The cartridge may be packaged together with one or several capsules of material to be used. The cartridge is preferably delivered and stored in sterilized packaging in a temperature controlled environment. For example, the cartridge can be a dry consumable in which the user places a capsule with a fluid. Alternatively, the cartridge can be pre-filled with an ink or a reagent.

The bioink may be being sold in a separate material capsule. Before starting the printing process the user may plug the capsule with ink into the cartridge. Optionally, the user can also plug the capsule with a reagent e.g. a neutralization solution. Optionally, the user also pipettes the cells in medium into one of the compartments.

The material (e.g. bioink) capsule may be made of a polymer, metal, glass, elastomer or a combination of those materials.

Bioink capsules might come in two different sizes, a larger capsule for bioink and a smaller capsule for reagents. Thus, material compartments may be adapted for different sized and/or configured capsules. The different sized capsules may also be supplied with different filled volume so depending on the printing protocol to be executed, proper capsules are selected.

The cartridge and/or a capsule may be made of a material that blocks light of certain wavelengths to prevent premature gelation of photo-crosslinkable polymers.

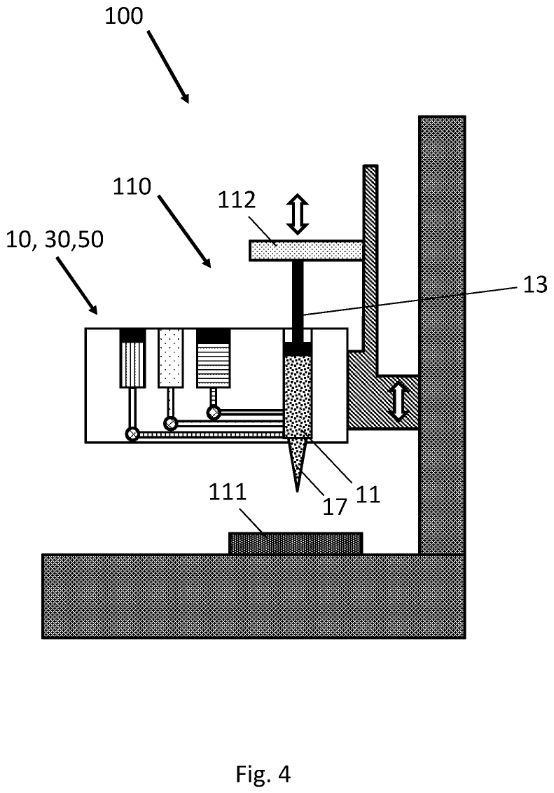

As mentioned, the material mixing cartridge is preferably adapted for mounting in a dispensing system. Herein is described an example of use in a bioprinter, as schematically illustrated in FIG. 4. A material mixing cartridge of any of the types described herein is mounted in a bioprinter 100. Preferably the bioprinter 100 has a dedicated cartridge station 110 for this purpose, suitably arranged above a printing region 111. A cartridge station 110 may be a movable part of the bioprinter, configured to move during the printing process. A printing region 11 may be a dedicated platform, adapted for supporting a petri dish or the like.

The bioprinter 100 preferably comprises a control unit adapted to control dispensing of material from the material mixing cartridge. The control unit of the bioprinter 100 may be configured to control the mixing and/or dispensing steps of the cartridge. Thus, in one aspect, the user first adds the desired materials to the material compartments in the cartridge, and thereafter places the cartridge in a cartridge station on the bioprinter 100. Optionally, a nozzle 17 is added to the cartridge. As an alternative or in combination, mixing is performed before mounting the cartridge, for example by manually actuating the piston 13 and actuating the appropriate valves 21.

Preferably just prior to initiating the printing (dispensing) process, the bioprinter 100 actuates the relevant parts of the cartridge as described above to perform mixing of the material. In one aspect, the control unit automatically controls transfer of materials within the material mixing cartridge. Thereafter, the control unit initiates dispensing of material through a nozzle 17 onto the printing region 111.

Mixing may include an initial step of transferring a first material, such as a bioink, from a material compartment to the dispensing chamber 11, thereafter transferring a neutralizing agent from one of the other material compartments to the dispensing chamber 11, to neutralize the bioink, and subsequently transferring cells in medium from a material compartment to the dispensing chamber 11. As an alternative, the mixing may include an initial step of transferring a first material, such as a bioink, from a material compartment to the dispensing chamber 11, followed by transferring cells in medium from another material compartment to the dispensing chamber 11.

During the mixing procedure, as well as during the dispensing procedure, the bioprinter may actuate the dispensing piston 13 by an actuating part 112, adapted to interact with the piston 13 when the cartridge is mounted in the bioprinter, e.g. my moving up and down.

In some aspects, the bioprinter and/or cartridge are configured to automatically initiate mixing upon connection of the cartridge to the cartridge station.

The cartridge and bioprinter may be equipped with a cartridge type detection system or arrangement, e.g. an electrical circuit, NFC chip, a mark that can be detected by a sensor inside the instrument or a magnetic part that can be detected by a sensor inside the instrument. Such a cartridge type detection system may be configured to automatically trigger mixing and/or dispensing in the cartridge.

The dispensing systems disclosed herein may further include temperature regulation means, in order to control the temperature of the material in the cartridge. The cartridge may further be pre-cooled by the dispensing instrument, or the user can be advised to keep the cartridge in pre-cooled condition prior to loading the material. In some aspects, the entire cartridge, or at least the dispensing chamber is temperature controlled by a temperature-regulated block at a dedicated cartridge station. Thus, the temperature may be regulated by thermoelectric elements mounted inside the dispensing instrument. The regulation may be performed by the control unit of the dispensing system and based on sensor feedback from the cooling block and a pre-validated thermal model of the cartridge. The model can be dependent on what printing protocol is performed and ambient temperature.

In any material mixing cartridge disclosed herein, channels between the compartments and dispensing chamber may contain fluid presence sensors that detect if the material is flowing in the channel. These sensors can be used to prime the channels to reduce the amount of air added to the mix.

Furthermore, a material mixing cartridge may contain fluid sensors that measures the volume of fluids within the compartments and/or capsules as well as the volume of cell media.

The disclosed material mixing cartridge makes it much more convenient to prepare the material for a bioprinting or a dispensing process. It saves time as well as removes the possibility for a human error. Precise control of the mixing speed in combination with the information on the volume and type of ink allows to select an optimal mixing speed that will improve cell viability over traditional methods. Furthermore, precise dosing of the neutralization solution and ink will assure the right pH of neutralized ink.

Periodical mixing of a bioink during the dispensing process e.g. between wells or between layers will resuspend the cells to assure similar cell counts between dispensed samples or in all parts of a printed model.

Since the mixing steps are performed inside a single use, sterilized consumables the invention also minimizes the possibility of contamination of the sample.

Because material mixing cartridges will be placed in a cartridge station built into a bioprinter or a biodispenser, all material preparation steps can be performed in a precisely controlled temperature. This is absolutely crucial to material preparation steps of some hydrogels e.g. collagen. In other workflows it is difficult to provide stable temperature conditions.

As mentioned, the cartridge may be equipped with a valve between a dispensing chamber 11 and the dispensing outlet 12. Such valve would prevent air from entering the chamber while transferring fluids into the dispensing chamber. It would also prevent leaking the fluid during the same operation.

For certain combinations of high viscosity material and small dispensing nozzle sizes the dispensing pressure might exceed the maximum limitations of the valving system. In such a scenario a series of passive valves can be used to allow higher pressures.

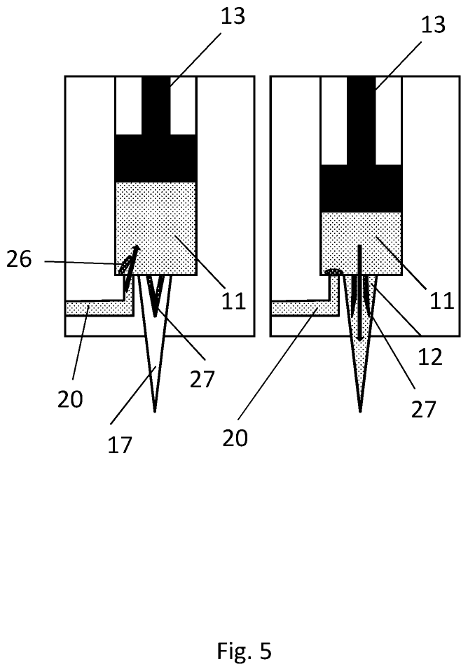

An example of a valve arrangement is schematically illustrated in FIG. 5. The left image illustrates the phase of transferring material from the transfer channel 20 to the dispensing chamber 11. Material is being pulled into the dispensing chamber through a transfer valve 26 by pulling piston 13 upwards. Notably, the transfer valve 26 may be a separate valve from the material compartment valves 21, or the same valve. A dispensing valve 27 is arranged in the dispensing outlet 12, and is arranged such that when there is a negative pressure in the dispensing chamber 11 the dispensing valve 27 will be closed, and thus no material is allowed to exit through the dispensing outlet 12.

In the right image of FIG. 5, piston 13 is actuated downwards, thereby creating an increased pressure in the dispensing chamber 11. Transfer valve 26 is thereby closed and dispensing valve opened to allow dispensing of material through the dispensing outlet 12. Notably, transfer valve 26 and dispensing valve 27 may be any type of suitable passive valve. As an alternative, a similar arrangement may be provided using active valves, either manually or automatically operated.

### EXAMPLES

In one example a capsule with a bioink, e.g. GelMA, is placed in the ink capsule compartment. Subsequently, the cells in cell medium are pipetted into the compartment for cells in medium. After the cartridge is loaded into the dispensing instrument, in this case a 3D bioprinter, the bioprinter causes the valving system to open the path between the compartments that contain fluids. Subsequently, the instrument forces the back-and-forth movement of the fluids through the narrow transfer channels causing the fluids to mix together. Alternatively, one of the fluids can be transferred to the dispensing chamber before the mixing process starts. After the ink has been mixed with cells and the bioink has been placed in the dispensing chamber, the bioprinter opens the dispensing valve and extrudes the fluid through the dispensing outlet.

In another example a capsule with a bioink, e.g. Collagen Premium, is placed in the ink capsule compartment. Subsequently, a capsule with a neutralization solution can be placed in the reagent capsule compartment. After the cartridge is loaded into the bioprinter, the ink is mixed with a neutralization solution to alter the pH of the ink. Optionally, the cartridge can be equipped with a pH sensor that measures pH of the ink before, during and after cells in cell medium are added. Subsequently, the ink is mixed with cells in medium. After the ink has been mixed with cells and the bioink has been placed in the dispensing chamber, the instrument opens the dispensing valve and extrudes the fluid through the dispensing outlet.

The present invention is not limited to the above-described preferred embodiments. Various alternatives, modifications and equivalents may be used. Therefore, the above embodiments should not be taken as limiting the scope of the invention, which is defined by the appending claims.

1. A material mixing cartridge for dispensing systems, comprising
a dispensing chamber, comprising a material dispensing outlet and a dispensing actuator for dispensing material through the material dispensing, outlet,
at least two material compartments for receiving material to be mixed, and
at least one material transfer channel being adapted for material transfer between said at least two material compartments and said dispensing chamber,
said material mixing cartridge further comprising at least one valve arranged to regulate material flow between a material compartment and the dispensing chamber via the at least one material transfer channel,
wherein the dispensing chamber, material compartments and at least one valve are configured such that material transfer between a material compartment and the dispensing chamber may be performed independently of dispensing material through the material dispensing outlet, and
wherein the dispensing chamber, material compartments and at least one valve are configured such that any material in the dispensing chamber may be transferred to any material compartment.

2. The material mixing cartridge according to claim 1, wherein the dispensing chamber, material compartments and at least one valve are configured such that material may be transferred from a material compartment to the dispensing chamber independently of dispensing material through said material dispensing outlet.

3. The material mixing cartridge according to claim 1, wherein the dispensing chamber, material compartments and at least one valve are configured such that material may be transferred from the dispensing chamber to a material compartment independently of dispensing material through said material dispensing outlet.

4. The material mixing cartridge according to claim 1, wherein at least one of the at least one valve is adapted to be movable between at least
i) a closed configuration,
ii) a configuration allowing material flow from the connected material compartment to the dispensing chamber, and
iii) a configuration allowing material flow from the dispensing chamber to the connected material compartment.

5. The material mixing cartridge according to claim 1, wherein each material compartment is connected to the material transfer channel via an individual valve for regulating flow between each respective material compartment and the dispensing chamber.

6. The material mixing cartridge according to claim 1, wherein two or more of the material compartments connect to a common material transfer channel connected to the dispensing chamber.

7. The material mixing cartridge according to claim 1, wherein the dispensing actuator is a dispensing piston adapted to dispense material in the dispensing chamber through the material outlet.

8. The material mixing cartridge according to claim 1, wherein at least one of the material compartments is adapted for receiving capsules of material to be mixed and said material compartment is further configured such that the received capsule is opened to the material transfer channel when the capsule is received in the material compartment.

9. The material mixing cartridge according to claim, claim 1, further comprising at least one transfer actuator adapted to provide pressure within at least one material compartment for transfer of material between said material compartment and said dispensing chamber.

10. The material mixing cartridge according to claim 1, further comprising a temperature regulation means for controlling temperature of the material in the cartridge.

11. The material mixing cartridge according to claim 1, further comprising an outer housing, wherein the dispensing chamber, material compartments, material transfer channel and at least one valve are contained within said outer housing.

12. The material mixing cartridge according to claim 1, wherein the dispensing system is a 3D bioprinter or a droplet dispensing system, and the material mixing cartridge is adapted for direct mounting in the dispensing system.

13. A dispensing system comprising
a material mixing cartridge according to claim 1,
a cartridge station adapted for mounting of said material mixing cartridge, and
a control unit adapted to control dispensing of material from the material mixing cartridge.

14. The dispensing system of claim 13, further comprising a cartridge type detection arrangement adapted to detect when a material mixing cartridge is mounted in the cartridge station.

15. The dispensing system of claim 13, wherein the control unit is further configured to automatically control transfer of materials within the material mixing cartridge and/or control dispensing of material from the dispensing chamber.

16. The dispensing system of any of claim 13, further comprising a temperature regulation means for controlling temperature of the material in the material mixing cartridge.

17. The dispensing system of any of claim 13, wherein the control unit is adapted to control
I. transferring material from the first material compartment to the dispensing chamber via the connected transfer channel,
II. subsequently or simultaneously transferring material from the second material compartment to the dispensing chamber via the connected transfer channel,
III transferring material from the dispensing chamber back to one of the material compartments and thereafter from said material compartment to the dispensing chamber via the transfer channel, and
IV. dispensing the mixed material from the dispensing chamber through the material dispensing outlet.

18. Method for mixing material to be dispensed in a dispensing system, said method comprising the steps of
a. adding material to a first material compartment in a material mixing cartridge for a dispensing system, said material compartment being connected to a dispensing chamber in said material mixing cartridge via a material transfer channel,
b. adding material to a second material compartment in said material mixing cartridge, said second material compartment also being connected to said dispensing chamber in said material mixing cartridge via a material transfer channel, said material mixing cartridge further comprising at least one valve arranged to regulate material flow between at least one of the material compartments and the dispensing chamber,
c. transferring material from the first material compartment to the dispensing chamber via the connected transfer channel,
d. subsequently or simultaneously transferring material from the second material compartment to the dispensing chamber via the connected transfer channel,
e. transferring material from the dispensing chamber back to one of the material compartments and thereafter from said material compartment to the dispensing chamber via the transfer channel,
f. dispensing the mixed material from the dispensing chamber through a material dispensing outlet.

19. Method according to claim 18, wherein adding material to a material compartment comprises arranging a capsule containing said material in said material compartment, and wherein upon arranging the capsule in said material compartment, the received capsule is opened to the material transfer channel.

20. Method according to aclaim 18, wherein after step b, a further step b1 is performed: b1 adding material to a third material compartment in said material mixing cartridge, said third material compartment also being connected to said dispensing chamber in said material mixing cartridge via a material transfer channel, and wherein said third material is transferred from the third material compartment to the dispensing chamber via the transfer channel any time before step f.

21. Method according to claim 18, wherein the transferal of material between at least one material compartment and the dispensing chamber is performed by aspiration via the material transfer channel.

22. Method according to claim 18, wherein the transferal of material between at least one material compartment and the dispensing chamber is performed by operating at least one of the at least one valves.
